(19) United States
(12) Patent Application Publication
(10) Pub. No.: US 2009/0306152 A1
(43) Pub. Date: Dec. 10, 2009

(54) COOLING COSMETIC OR DERMATOLOGICAL PREPARATIONS COMPRISING (1R,2S,5R)-2-ISOPROPYL-5-METHYL-N-(2-(PYRIDIN-2-YL)ETHYL)-CYCLOHEXANE CARBOXAMIDE AND/OR (1R,2S,5R)-N-(4-CYANOMETHYL-PHENYL)-2-ISOPROPYL-5-METHYLCYCLOHEXANE CARBOXAMIDE IN COMBINATION WITH MENTHOXYPROPANEDIOL

(75) Inventors: Ludger Kolbe, Dohren (DE); Julia Eckert, Hamburg (DE); Gitta Neufang, Hamburg (DE); Michael Woehrmann, Hamburg (DE); Stefanie Knaupmeier, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG., Hamburg (DE)

(21) Appl. No.: 12/402,985

(22) Filed: Mar. 12, 2009

(30) Foreign Application Priority Data
Mar. 20, 2008 (DE) 10 2008 015 424.5

(51) Int. Cl. A61K 31/4402; A61K 31/167; A61K 8/49; A61K 8/42; A61Q 19/00

(52) U.S. Cl. 514/357; 514/613

(57) ABSTRACT

A cooling topical cosmetic or dermatological preparation which comprises (1R,2S,5R)-2-isopropyl-5-methyl-N-(2-(pyridin-2-yl)ethyl)cyclohexane carboxamide and/or (1R,2S,5R)-N-(4-cyanomethyl-phenyl)-2-isopropyl-5-methylcyclohexane carboxamide, in combination with menthoxypropanediol.

### CROSS-REFERENCE TO RELATED APPLICATIONS

The present application claims priority under 35 U.S.C. § 119 of German Patent Application No. 10 2008 015 424.5, filed Mar. 20, 2008, the entire disclosure of which is expressly incorporated by reference herein.

### BACKGROUND OF THE INVENTION

1. Field of the Invention

The present invention relates to cooling cosmetic and dermatological preparations, in particular skin care cosmetic and dermatological preparations.

2. Discussion of Background Information

The cooling of the skin can be something very pleasant. This in particular if the skin feels hot, hurts, itches or is freshly shaven. This is often connected to sunburn or sunbathing. In the case of insect bites or inflamed skin, acne, cooling of the skin can likewise be felt as soothing.

There have hitherto been various cooling substances that evoke a pleasant feeling of freshness on the skin. For example, menthol is used in order to cause a feeling of freshness in after shave formulations. However, menthol has properties irritating to the mucous membranes and can cause further skin irritations, which leads to a burning of the skin. Furthermore, menthol has a strong inherent odor and can lead to intolerances in the eye region.

There has been no lack of attempts to cause the cooling of the skin by applying a preparation. There have also been attempts to find preparations that cool.

This has been tried in particular for skin after shaving or the skin after sunburn or sunbathing. Known products are called after shave or after sun.

It would be advantageous to overcome the disadvantages of the prior art and to provide preparations rendering possible a cooling of the skin, which are simple to produce, do not have an irritating effect on the skin or mucous membranes, have a neutral odor and provide a pleasant cooling with correct application.

### SUMMARY OF THE INVENTION

The present invention provides a cooling topical cosmetic or dermatological preparation which comprises a combination of (A) (i) (1R,2S,5R)-2-isopropyl-5-methyl-N-(2-(pyridin-2-yl)ethyl)-cyclohexane carboxamide and/or (ii) (1R,2S,5R)-N-(4-cyanomethyl-phenyl)-2-isopropyl-5-methylcyclohexane carboxamide, with (B) menthoxypropanediol.

In one aspect, the preparation may comprise from about 0.001% to about 10% by weight of (A), based on the total weight of the preparation. For example, the preparation may comprise at least about 0.01%, e.g., at least about 0.05% by weight of (A) and/or not more than about 1% by weight of (A).

In another aspect of the preparation, the weight ratio (A):(B) may be from about 1:1 to about 1:20, e.g., from about 1:1.5 to about 1:15. For example, the weight ratio (A):(B) may be not lower than about 1:3 and/or not higher than about 1:10.

In yet another aspect, the preparation may further comprise one or more antioxidants and/or one or more vitamins and/or one or more alcohols and/or one or more antiperspirants and/or one or more deodorants.

In a still further aspect, the preparation of the present invention may be present as an emulsion.

The present invention also provides a cooling topical cosmetic or dermatological preparation which comprises from about 0.01% to about 1% by weight of (A) (1R,2S,5R)-2-isopropyl-5-methyl-N-(2-(pyridin-2-yl)ethyl)cyclohexane carboxamide and/or (1R,2S,5R)-N-(4-cyanomethyl-phenyl)-2-isopropyl-5-methylcyclohexane carboxamide, in combination with (B) menthoxypropanediol in an amount such that the weight ratio (A):(B) is from about 1:1.5 to about 1:15.

The present invention also provides a method of creating a cooling sensation on the (human) skin. The method comprises applying to the skin the preparation according to the present invention as set forth above (including the various aspects thereof).

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice. Unless stated otherwise, all amounts, fractions and percentages given are based on the weight and the total amount or on the total weight of the preparations.

It was surprising and not foreseeable to one skilled in the art that cooling topical cosmetic or dermatological preparations comprising (A): (1R,2S,5R)-2-isopropyl-5-methyl-N-(2-(pyridin-2-yl)ethyl)-cyclohexane carboxamide and/or (1R,2S,5R)-N-(4-cyanomethyl-phenyl)-2-isopropyl-5-methylcyclohexane carboxamide in combination with (B): menthoxypropanediol overcome the disadvantages of the prior art.

Surprisingly, the combination (1R,2S,5R)-2-isopropyl-5-methyl-N-(2-(pyridin-2-yl)ethyl)-cyclohexane carboxamide

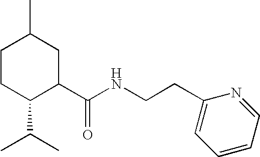

and/or (1R,2S,5R)-N-(4-cyanomethyl-phenyl)-2-isopropyl-5-methylcyclohexane carboxamide

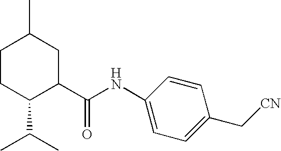

in combination with menthoxypropanediol

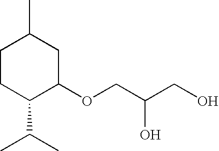

(cooling substances according to the present invention) leads to a synergistic cooling effect.

These cooling substances that reduce itching can also be used in combination with additional cooling substances. Non-limiting examples of additional cooling substances include menthol, menthone, ethyl menthane carboxamido acetate (WS-5), isopulegol (Coolact P), menthanediol (Coolact 38D), N,2,3-trimethyl-2-isopropylbutanamide (WS-23), ethyl menthane carboxamide (WS-3), menthone glycerine acetal (Frescolat MGA), mono-menthyl succinate (Physcool) or methyl ester of 2-isopropyl-5-methyl-cyclohexane carbonyl-amino-propionic acid.

The preparations according to the present invention are simple to formulate and do not put any great demands on the production processes.

The production of an emulsion and in particular, an O/W emulsion is preferred. To this end, the cooling substances may, for example, be predissolved in one or more alcohols (in particular, polyols) and added to the emulsion at about 40° C. before cooling to room temperature.

The present invention also comprises the use of (A) (1R,2S,5R)-2-isopropyl-5-methyl-N-(2-(pyridin-2-yl)ethyl)-cyclohexane carboxamide and/or (1R, 2S, 5R)-N-(4-cyanomethyl-phenyl)-2-isopropyl-5-methylcyclohexane carboxamide in combination with (B) meNthoxypropanediol for producing a feeling of coolness on the skin.

It is preferred for the preparations of the present invention to comprise from about 0.001% to about 10% by weight, particularly preferably from about 0.01% to about 1% by weight of (A), for example, not less than about 0.05% by weight, or not less than about 0.1% by weight, based on the total weight of the preparation. It also is preferred for the weight ratio (A):(B) to be from about 1:1 to about 1:20, particularly from about 1:1.5 to about 1:15. For example, the ratio may be at least about 1:2, e.g., at least about 1:3, or at least about 1:4 and/or not higher than about 1:12, e.g. not higher than about 1:10, or not higher than about 1:8. Further, when both (1R,2S,5R)-2-isopropyl-5-methyl-N-(2-(pyridin-2-yl)ethyl)-cyclohexane carboxamide and (1R,2S,5R)-N-(4-cyanomethyl-phenyl)-2-isopropyl-5-methylcyclohexane carboxamide are present, their weight ratio preferably is from about 20:1 to about 1:20, e.g., not lower than about 1:10 and/or not higher than about 10:1.

It is furthermore preferred for the preparations of the present invention to comprise one or more substances selected from antioxidants, vitamins, alcohols, antiperspirants, and deodorants.

The cosmetic and dermatological preparations according to the present invention may be applied to the skin and/or the hair in sufficient quantities in the manner usual for cosmetics.

The cosmetic and dermatological preparations according to the present invention may further comprise one or more cosmetic auxiliaries which are customarily used in such preparations, e.g., preservatives, bactericides, perfumes, substances for preventing or increasing foaming, dyes, pigments which have a coloring action, thickeners, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

An additional content of conventional antioxidants is generally preferred. According to the invention, favorable antioxidants which may be used include all antioxidants which are customary or suitable for cosmetic and/or dermatological applications.

The amount of antioxidants (one or more compounds) in the preparations of the present invention is preferably from about 0.001% to about 30% by weight, particularly preferably from about 0.05% to about 20% by weight, in particular from about 0.1% to about 10% by weight, based on the total weight of the preparation.

If the cosmetic or dermatological preparation of the present invention is present as a solution or emulsion or dispersion, the following substances may, for example, be used as solvent:
water or aqueous solutions
oils such as triglycerides of capric or caprylic acid, but preferably castor oil;
fats, waxes and other natural and synthetic fatty bodies, preferably esters of fatty acids with alcohols of low carbon number, e.g., with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular mixtures of the above-mentioned solvents may be used. In the case of alcoholic solvents, water can be a further constituent.

As propellant for cosmetic and/or dermatological preparations of the present invention that are sprayable from aerosol containers the usual known volatile liquefied propellants, for example hydrocarbons (propane, butane, isobutane) are suitable which can be used alone or mixed with one another. Compressed air can also be used advantageously.

One skilled in the art will, of course, be familiar with the fact that there are non-toxic propellants, which would be suitable in principle for putting into practice the present invention in the form of aerosol preparations, but use thereof is not recommended—in particular fluorohydrocarbons and fluorochlorohydrocarbons (FCHCs)—due to their unacceptable effect on the environment or other accompanying circumstances.

Cosmetic preparations in accordance with the present invention may also be present as gels which comprise not only an effective amount of active ingredient according to the invention and conventionally used solvents therefor, preferably water, but also organic thickeners, for example gum arabic, xanthan gum, sodium alginate, cellulose derivatives, preferably methylcellulose, hydroxymethylcellulose, hydroxyethylcellulose, hydroxypropylcellulose, hydroxypropylmethylcellulose, or inorganic thickeners, for example aluminum silicates such as, for example, bentonites, or a mixture of polyethylene glycol and polyethylene glycol stearate or polyethylene glycol distearate. The gel may comprise the thickener in an amount of, for example, from about 0.1% to about 30% by weight, preferably from about 0.5% to about 15% by weight.

The preparation according to the present invention is preferably present in a form which is suitable for topical application. For example, the composition may be present in the form of a cream, a lotion, a gel, an ointment, a tincture, a skin oil, a milk, a balm, a bandage impregnated with the preparation, a cloth impregnated with the preparation, a textile impregnated with the preparation, a pad impregnated with the preparation, a spray, an aerosol, a roll-on, a stick, a soft solid, a powder or a powder spray.

Particularly advantageous preparations are furthermore obtained if antioxidants are used as additives or active ingredients. Water-soluble antioxidants can be used very particularly advantageously, such as, for example, vitamins, e.g., ascorbic acid and derivatives thereof—in particular ascorbylpalmitate, Na and Mg ascorbylphosphate and ascorbylacetate—as well as rutinic acid and derivatives thereof in particular alpha-glucosylrutin, quercetin and isoquercetin.

Examples of particularly preferred antioxidants include furthermore vitamin E and derivatives thereof (in particular vitamin E acetate), vitamin A and derivatives thereof (in particular vitamin A palmitate), and camosine, butylated hydroxytoluol, butylated hydroxyanisol, beta-alanine, as well as carotenoids (in particular beta-carotene) and phytoene.

The concentration of the antioxidants (one or more compounds) in the preparations is preferably from about 0.001% to about 30% by weight, particularly preferably from about 0.05% to about 20% by weight, in particular from about 1% to about 10% by weight, based on the total weight of the preparation.

It may also be advantageous if the preparations according to the present invention comprise oil-soluble UVA filters and/or UVB filters in the lipid phase and/or water-soluble UVA filters and/or UVB filters in the aqueous phase.

Sunscreen containing formulations according to the present invention may advantageously comprise other substances which absorb UV radiation in the UVA and/or UVB range, the total amount of the filter substances being, for example, from about 0.1% to about 30% by weight, preferably from about 0.5% to about 10% by weight, in particular from about 1% to about 6% by weight, based on the total weight of the preparations, in order to provide cosmetic preparations that protect the skin from the entire range of ultraviolet radiation. They can also serve as sunscreen agents.

The following examples are intended to illustrate the present invention without restricting it. All quantities, proportions and percentages are based on the weight and the total quantity or on the total weight of the preparations unless stated otherwise.

| | % by |
|---|---|
| Day cream | weight |
| Glyceryl stearate citrate | 3 |
| Cetyl alcohol | 2 |
| Glyceryl stearate | 1 |
| Cyclomethicone | 3 |
| Butylmethoxydibenzoylmethane (Parsol ® 1789) | 2 |
| Ethylhexyl-2-cyano-3,3-diphenylacrylate (Octocrylene) | 5 |
| Ethylhexyl methoxycinnamate | 2 |
| Menthoxypropanediol | 1 |
| (1R,2S,5R)-2-Isopropyl-5-methyl-N-(2-(pyridin-2-yl)ethyl)- | 0.1 |
| cyclohexancarboxamide | |
| C₁₂₋₁₅ Alkyl benzoate | 3 |
| Carbomer | 0.2 |
| Sodium ascorbylphosphate | 0.2 |
| 3-(4-Hydroxy-3-methoxyphenyl)-1-(4-hydroxyphenyl)- | 0.5 |
| propane-1-one | |
| Glycerin | 8 |
| (1R,2S,5R)-N-(4-Cyanomethyl-phenyl)-2-isopropyl-5-methyl | 0.02 |
| cyclohexane carboxamide | |
| Perfume | 0.3 |
| Aqueous NaOH | q.s. |
| Water | ad 100 |

pH 5

| | % by |
|---|---|
| Skin care cream | weight |
| Glyceryl stearate, self-emulsifying | 5 |
| Stearyl alcohol | 3 |
| Stearic acid | 1 |
| (1R,2S,5R)-N-(4-Cyanomethyl-phenyl)-2-isopropyl-5- | 0.08 |
| methyl cyclohexane carboxamide | |
| Dimethiconol | 0.5 |
| Cyclomethicone | 4 |
| Octyldodecanol | 3 |
| Hydrogenated cocoglycerides | 2 |
| Menthoxypropanediol | 0.4 |
| 3-(4-Hydroxy-3-methoxyphenyl)-1-(4-hydroxyphenyl)- | 0.2 |
| propane-1-one | |
| Sodium starch octenylsuccinate | 1 |
| Carbomer | 0.2 |
| (1R,2S,5R)-2-Isopropyl-5-methyl-N-(2-(pyridin-2- | 0.06 |
| yl)ethyl)-cyclo-hexane carboxamide | |
| Glycerin | 9 |
| EDTA | 0.1 |
| Perfume | 0.3 |
| Tocopheryl acetate | 0.5 |
| Coenzyme Q10 | 0.1 |
| Methylpropanediol | 1 |
| Aqueous NaOH | q.s. |
| Water | ad 100 |

pH: 7

| | % by |
|---|---|
| Cooling skin cream | weight |
| Polyglyceryl-3 methylglucose distearate: | 2 |
| Sorbitan stearate | 2 |
| Stearylalcohol | 2 |
| Dimethicone | 2 |
| C₁₂₋₁₅ Alkylbenzoate | 2 |
| Caprylic acid/capric acid triglyceride | 3 |
| Cyclomethicone | 4 |
| Ethylhexyl triazone | 1 |
| 2,2′-Methylene-bis-(6-(2H-benzotriazol-2-yl)-4- | 2 |
| (1,1,3,3-tetramethyl butyl)-phenol) | |
| Ethylhexyl-2-cyano-3,3-diphenylacrylate (Octocrylene) | 3 |
| 3-(4-Hydroxy-3-methoxyphenyl)-1-(4-hydroxyphenyl)- | 0.5 |
| propane-1-one | |
| Carrageenan | 0.1 |
| Crosslinked alkylacrylate (alkylacrylate crosspolymer) | 0.1 |
| (1R,2S,5R)-N-(4-Cyanomethyl-phenyl)-2-isopropyl-5-methyl- | 0.05 |
| cyclohexane carboxamide | |
| Menthoxypropanediol | 0.8 |
| Glycerin | 8 |
| Ethylhexylglycerin | 0.4 |
| (1R,2S,5R)-2-Isopropyl-5-methyl-N-(2-(pyridin-2- | 0.06 |
| yl)ethyl)-cyclohexane carboxamide | |
| Ethylparaben | 0.2 |
| Phenoxyethanol | 0.4 |
| Additive (nylon, starch phosphate) | 2 |
| Citric acid, sodium salt | q.s. |
| Water | ad 100 |

pH: 6

| | % by |
|---|---|
| Sunscreen cream with cooling effect | weight |
| Glyceryl stearate | 3 |
| PEG-40 stearate | 1 |
| Cetyl alcohol | 2 |
| C₁₂₋₁₅ Alkylbenzoate | 2 |
| Dicaprylylether | 2 |
| Octyldodecanol | 2 |
| Cyclomethicone | 2 |
| Myristyl myristate | 1 |
| (1R,2S,5R)-N-(4-Cyanomethyl-phenyl)-2-isopropyl-5-methyl | 0.04 |
| cyclohexane carboxamide | |
| Ethylhexyl methoxycinnamate | 6 |
| Phenylbenzimidazole sulfonic acid | 1 |
| Aniso Triazin (2,4-bis-{[4-(2-ethyl-hexyloxy)-2- | 2 |
| hydroxy]-phenyl}-6-(4-methoxyphenyl)-1,3,5-triazine) | |
| 3-(4-Hydroxy-3-methoxyphenyl)-1-(4-hydroxyphenyl)- | 0.2 |
| propane-1-one | |
| Carragenan | 0.2 |
| Carbomer | 0.2 |
| Glycerin | 7 |
| Butylene glycol | 3 |
| (1R,2S,5R)-2-Isopropyl-5-methyl-N-(2-(pyridin-2-yl)ethyl)- | 0.6 |
| cyclohexane carboxamide | |
| Menthoxypropanediol | 1.5 |
| Perfume | q.s. |
| Aqueous NaOH | q.s. |
| Water | ad 100 |

pH: 7

| | % by |
|---|---|
| After Shave Balm | weight |
| Triceteareth-4 phosphate | 1 |
| Cyclomethicone | 3 |
| C₁₂₋₁₅ Alkylbenzoate | 2 |
| Menthoxypropanediol | 1 |
| Distarch phosphate | 1 |
| (1R,2S,5R)-2-Isopropyl-5-methyl-N-(2-(pyridin- | 0.1 |
| 2-yl)ethyl)-cyclohexane carboxamide | |
| Xanthan Gum | 0.1 |
| Methylparaben | 0.2 |
| Phenoxyethanol | 0.4 |
| (1R,2S,5R)-N-(4-Cyanomethyl-phenyl)-2- | 0.04 |
| isopropyl-5-methyl-cyclohexane carboxamide | |
| Glycerin | 6 |
| EDTA | 0.1 |
| Perfume | 0.3 |
| Aqueous NaOH | q.s. |
| Water | ad 100 |

pH: 7

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cooling topical cosmetic or dermatological preparation, wherein the preparation comprises
(A) at least one of (i) (1R,2S,5R)-2-isopropyl-5-methyl-N-(2-(pyridin-2-yl)ethyl)-cyclohexane carboxamide and (ii) (1R,2S,5R)-N-(4-cyanomethyl-phenyl)-2-isopropyl-5-methylcyclohexane carboxamide, and
(B) menthoxypropanediol.

2. The preparation of claim 1, wherein the preparation comprises from about 0.001% to about 10% by weight of (A), based on a total weight of the preparation.

3. The preparation of claim 2, wherein the preparation comprises at least about 0.01% by weight of (A).

4. The preparation of claim 2, wherein the preparation comprises at least about 0.05% by weight of (A).

5. The preparation of claim 2, wherein the preparation comprises not more than about 1% by weight of (A).

6. The preparation of claim 1, wherein a weight ratio (A):(B) is from about 1:1 to about 1:20.

7. The preparation of claim 1, wherein a weight ratio (A):(B) is from about 1:1.5 to about 1:15.

8. The preparation of claim 1, wherein a weight ratio (A):(B) is not lower than about 1:3.

9. The preparation of claim 1, wherein a weight ratio (A):(B) is not higher than about 1:10.

10. The preparation of claim 1, wherein the preparation further comprises at least one antioxidant.

11. The preparation of claim 1, wherein the preparation further comprises at least one vitamin.

12. The preparation of claim 1, wherein the preparation further comprises at least one alcohol.

13. The preparation of claim 1, wherein the preparation further comprises at least one antiperspirant.

14. The preparation of claim 1, wherein the preparation further comprises at least one deodorant.

15. The preparation of claim 1, wherein the preparation is present as an emulsion.

16. The preparation of claim 1, wherein the preparation comprises at least (A)(i).

17. The preparation of claim 1, wherein the preparation comprises at least (A)(ii).

18. The preparation of claim 1, wherein the preparation comprises both (A)(i) and (A) (ii).

19. A cooling topical cosmetic or dermatological preparation, wherein the preparation comprises
from about 0.01% to about 1% by weight of (A) at least one of (i) (1R,2S,5R)-2-isopropyl-5-methyl-N-(2-(pyridin-2-yl)ethyl)-cyclohexane carboxamide and (ii) (1R,2S,5R)-N-(4-cyanomethyl-phenyl)-2-isopropyl-5-methylcyclohexane carboxamide, and
(B) menthoxypropanediol,
a weight ratio (A):(B) being from about 1:1.5 to about 1:15.

20. A method of producing a cooling sensation on skin, wherein the method comprises applying to the skin the preparation of claim 1.
